(19) United States
(12) Patent Application Publication
(10) Pub. No.: US 2018/0132819 A1
(43) Pub. Date: May 17, 2018

(54) INTRA-CARDIAC ECHOCARDIOGRAPHY WITH MAGNETIC COUPLING

(71) Applicant: Mayo Foundation for Medical Education and Research, Rochester, MN (US)

(72) Inventor: Peter M. Pollak, Atlantic Beach, FL (US)

(73) Assignee: Mayo Foundation for Medical Education and Research, Rochester, MN (US)

(21) Appl. No.: 15/574,666

(22) PCT Filed: May 18, 2016
(86) PCT No.: PCT/US2016/033008

§ 371 Date: Nov. 16, 2017

Related U.S. Application Data
(60) Provisional application No. 62/163,073, filed on May 18, 2015.

(51) Int. Cl. A61B 8/12; A61B 8/08; A61B 8/00; A61B 17/34; A61B 34/00

(52) U.S. Cl. CPC A61B 8/12; A61B 8/0883; A61B 8/445; A61B 2034/2063; A61B 34/73; A61B 8/0841; A61B 2017/3413; A61B 17/3478

(57) ABSTRACT

This document provides devices and methods for interventional treatment of heart conditions. For example, this document provides devices and methods that enhance intra-cardiac echocardiography visualization of interventional devices such as trans-septal puncturing needles.

### CROSS-REFERENCE TO RELATED APPLICATIONS

This application claims the benefit of U.S. Provisional Application Ser. No. 62/163,073, filed May 18, 2015. The disclosure of the prior application is considered part of (and is incorporated by reference in) the disclosure of this application.

### BACKGROUND

### 1. Technical Field

This document relates to devices and methods for interventional treatment of heart conditions. For example, this document relates to devices and methods that enhance intra-cardiac echocardiography visualization of interventional devices such as, but not limited to, trans-septal puncturing needles.

### 2. Background Information

The left atrium (LA) is perhaps the most difficult cardiac chamber to access percutaneously. A trans-septal puncture (TSP) procedure using a puncturing needle permits a direct route to the LA via the systemic venous system and interatrial septum.

Direct visualization of the intracardiac anatomy and trans-septal puncturing needle during the TSP is advantageous. One method for such direct visualization is using intra-cardiac echocardiography (ICE).

A common challenge when using ICE during a TSP procedure is to consistently maintain appropriate alignment between the trans-septal puncturing needle and the ICE ultrasound beam so that the needle remains visible throughout the duration of the TSP procedure.

### SUMMARY

This document provides devices and methods for interventional treatment of heart conditions. For example, this document provides devices and methods that enhance intra-cardiac echocardiography visualization of interventional devices such as trans-septal puncturing needles.

In one implementation, a medical device system includes an intra-cardiac echocardiography (ICE) system, and a second medical device. A distal end portion of the ICE system comprises an ICE system coupler. A portion of the second medical device comprises a complementary coupler that is configured and operable to releasably couple with the ICE system coupler. The coupling between the ICE system coupler and the complementary coupler results in an orientation therebetween that facilitates a desired in-situ visualization of the second medical device by the ICE system.

Such a medical device system may optionally include one or more of the following features. The ICE system coupler may be attached to an ultrasonic probe of the ICE system. The ICE system coupler may be attached to a delivery sheath of the ICE system. The second medical device may comprise a trans-septal puncturing needle system. The complementary coupler may be attached to a needle of the trans-septal puncturing needle system. The complementary coupler may be attached to a delivery sheath of the trans-septal puncturing needle system. While the ICE system and trans-septal puncturing needle system are coupled together via the ICE system coupler and the complementary coupler, at least a distal tip of a needle of the trans-septal puncturing needle system may be maintained in a position to be consistently visible by the ICE system. While the ICE system and trans-septal puncturing needle system are coupled together via the ICE system coupler and the complementary coupler, essentially an entire distal end portion of the trans-septal puncturing needle system is maintained in a position to be consistently visible by the ICE system. The ICE system coupler and the complementary coupler may be attracted to each other by magnetism. One or both of the ICE system coupler and the complementary coupler may comprise a permanent magnet. The ICE system coupler and the complementary coupler may comprise mating mechanical structures. The ICE system coupler and the complementary coupler may comprise mating mechanical structures whereby the ICE system coupler and the complementary coupler are releasably coupleable with each other.

In another implementation, a method for in-situ releasable coupling of an intra-cardiac echocardiography (ICE) system and a second medical device includes (a) navigating, using an imaging modality, the ICE system such that a distal end portion of the ICE system is located within a target location within a patient, wherein a distal end portion of the ICE system comprises an ICE system coupler; (b) navigating, using an imaging modality, the second medical device such that a distal end portion of the second medical device system is located within the target location within the patient, wherein a portion of the second medical device comprises a complementary coupler that is configured and operable to releasably couple with the ICE system coupler; and (c) coupling, while the distal end portion of the ICE system and the distal end portion of the second medical device system are both located within the target location within the patient, the ICE system coupler with the complementary coupler such that the coupling results in an orientation therebetween that facilitates a desired in-situ visualization of the second medical device by the ICE system.

Such a method for in-situ releasable coupling of an intra-cardiac echocardiography (ICE) system and a second medical device may optionally include one or more of the following features. The second medical device may comprise a trans-septal puncturing needle system. The method may further comprise puncturing, using the trans-septal puncturing needle system, an interatrial septum of the patient while under direct visualization by the ICE system.

Particular embodiments of the subject matter described in this document can be implemented to realize one or more of the following advantages. Consistent direct visualization of a trans-septal puncturing needle during an interatrial septum puncture procedure can be facilitated using the devices and methods provided herein. As such, in some scenarios the trans-septal puncture procedure can be made safer and more effective. In addition, the devices and methods provided herein facilitate trans-septal puncture procedures in difficult cases such as, in instances of previous failures, unusual anatomy, the presence of prosthetic material, or when the patient has a therapeutic international normalized ratio. In some embodiments, various heart conditions can be treated using electrophysiology and/or interventional procedures facilitated by the devices and methods provided herein. Such heart conditions can be treated in a minimally invasive fashion using the devices and methods provided herein. Minimally invasive techniques can reduce recovery times, patient discomfort, and treatment costs.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims.

### DESCRIPTION OF THE DRAWINGS

FIG. 1 is a schematic diagram of cutaway human heart undergoing a TSP procedure using direct visualization via ICE in accordance with some embodiments provided herein.

FIG. 2 is a side view of distal end portions of an ICE probe and a trans-septal puncturing needle that include complementary magnetic coupling collars in accordance with some embodiments provided herein. The ICE probe and a trans-septal puncturing needle are decoupled in this view.

FIG. 3 is a top view of the decoupled ICE probe and trans-septal puncturing needle in an arrangement corresponding to FIG. 2.

FIG. 4 is a side view of distal end portions of the ICE probe and the trans-septal puncturing needle of FIG. 2. The ICE probe and a trans-septal puncturing needle are coupled in this view.

FIG. 5 is a top view of the coupled ICE probe and trans-septal puncturing needle in an arrangement corresponding to FIG. 4.

Like reference numbers represent corresponding parts throughout.

### DETAILED DESCRIPTION

This document provides devices and methods for interventional treatment of heart conditions. For example, this document provides devices and methods that enhance ICE visualization of interventional devices such as trans-septal puncturing needles. In addition to enhancing TSP procedures as described further below, it is envisioned that the devices and methods provided herein can be beneficially applied in conjunction with other procedures such as, but not limited to, evaluation of intracardiac thrombus, atrial septal defect/patent foramen ovale closure, interventional electrophysiological procedures, pulmonary vein ablation in patients with atrial fibrillation, atrial flutter ablation, ventricular tachycardia ablation, diagnosis/biopsy of intracardiac masses, balloon mitral valvuloplasty, atrial appendage occlusion, and visualization of the coronary sinus. Such implementations are also within the scope of this disclosure.

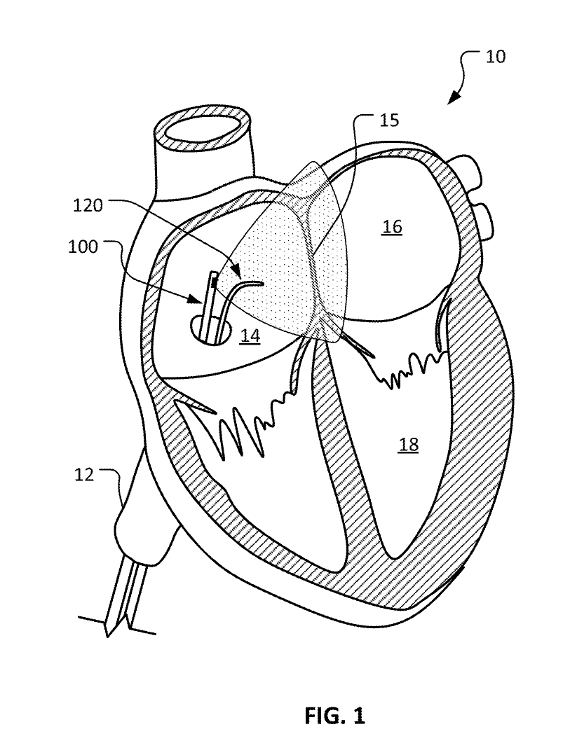

Referring to FIG. 1, a human heart 10 is depicted as undergoing a transcatheter interventional procedure. In this illustrative example, the procedure being performed is a TSP (trans-septal puncture) procedure. Namely, a trans-septal puncturing needle system 120 is being used to puncture an interatrial septum 15. To arrive at the arrangement shown, trans-septal puncturing needle system 120 has been advanced through the patient's vasculature to the inferior vena cava 12, and into the right atrium 14. Fluoroscopy is often used to facilitate the navigation of such catheter devices throughout the patient's anatomy. From its position in right atrium 14, trans-septal puncturing needle system 120 will be used to puncture interatrial septum 15 to thereby gain access to the left ventricle 16.

It should be understood that the TSP procedure is merely one exemplary procedure that can advantageously incorporate the magnetic coupling devices and methods provided herein. It is envisioned that the aforementioned other procedures, without limitation, can also use devices that are adapted to include the magnetic coupling mechanisms and concepts described herein. Such additional devices and procedures are also within the scope of this disclosure.

The exemplary TSP procedure is being performed using direct visualization via an ICE (intra-cardiac echocardiography) system 100. ICE system 100 is used by a clinician (e.g., interventional cardiologist) to facilitate a safe and effective TSP procedure. For example, in some cases the clinician will use ICE system 100 to facilitate the interatrial septum puncture through the fossa ovalis of interatrial septum 15. In such a case, ICE system 100 can be used to visualize the orientation of puncturing needle system 120 in relation to interatrial septum 15.

In some embodiments, trans-septal puncturing needle system 120 includes a curved transseptal introducer (sheath) and a curved needle within the introducer that is used to make the transseptal puncture after the introducer is used to guide the needle into position. In particular, once the transseptal introducer is in the right atrium 14, the distal tip of the guiding introducer is positioned against a puncture site, such as the fossa ovalis in inter-atrial septal wall 15. The needle (e.g., Brockenbrough needle) is then advanced distally through the transseptal introducer beyond the distal end of the introducer until it punctures the fossa ovalis. If the introducer includes a dilator, the dilator may be advanced with a needle through the punctured fossa ovalis to prepare an access port through the septum 15 and into the left atrium 16. Once the sheath has been seated across the septum and in the left atrium, the dilator, if present, and the needle may be withdrawn from the sheath. This sheath then provides lumenal access into the left atrium 16 for direct insertion of, for example, a treatment or diagnostic catheter.

While in some circumstances ICE system 100 can be used beneficially to visualize the position and/or orientation of puncturing needle system 120, ICE system 100 is limited to a planar field of view. Accordingly, and because ICE system 100 is separated from puncturing needle system 120, some or all portions of puncturing needle system 120 may not be consistently located within the planar field of view of ICE system 100. As such, a clinician may find it challenging to reap all the benefits that ICE system 100 can potentially provide for visualization during such procedures.

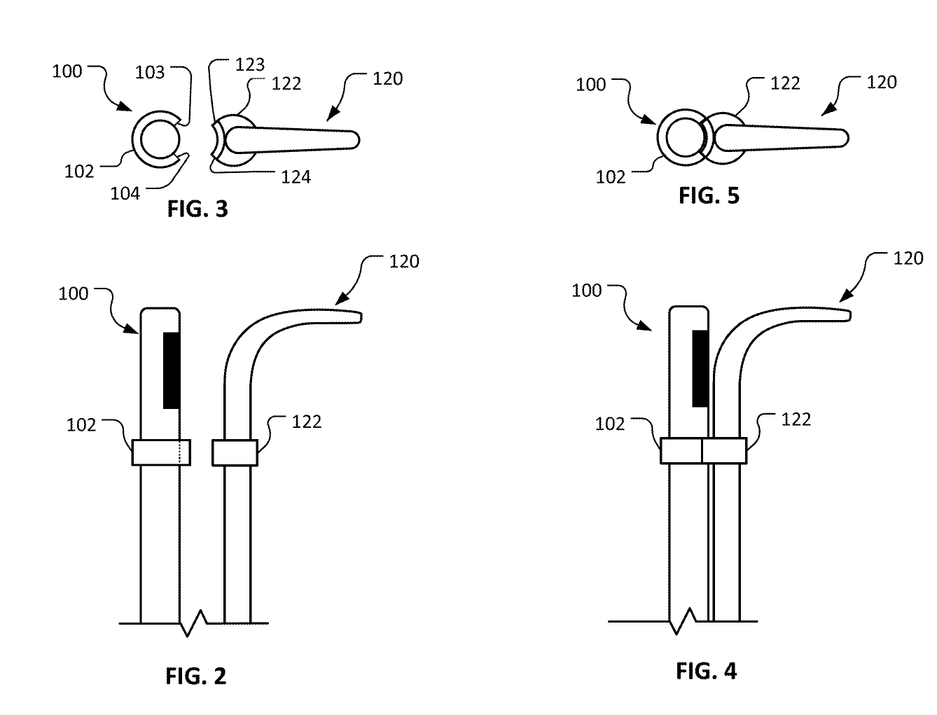

Referring also to FIGS. 2 and 3, in accordance with some aspects of the inventive disclosure provided herein, ICE system 100 and trans-septal puncturing needle system 120 can be adapted to couple with each other in-situ (e.g., in right atrium 14. Such coupling can result in ICE system 100 being oriented in relation to trans-septal puncturing needle system 120 such that essentially all distal end portions of trans-septal puncturing needle system 120 are advantageously within the field of view of ICE system 100.

In the depicted embodiment, ICE system 100 includes an ICE system coupler 102 and trans-septal puncturing needle system 120 includes a complementary needle system coupler 122. In some embodiments, ICE system coupler 102 is attached to the ultrasonic probe of ICE system 100. Alternatively or additionally, in some embodiments ICE system coupler 102 is attached to the delivery sheath of ICE system 100. Likewise, in some embodiments needle system coupler 122 is attached to the needle of trans-septal puncturing needle system 120. Alternatively or additionally, in some embodiments needle system coupler 122 is attached to the delivery sheath of trans-septal puncturing needle system 120. In any arrangement, the coupling between ICE system coupler 102 and complementary needle system coupler 122 results in ICE system 100 being oriented in relation to trans-septal puncturing needle system 120 such that essentially all distal end portions of trans-septal puncturing needle system 120 are advantageously within the field of view of ICE system 100.

In some embodiments, ICE system coupler 102 and needle system coupler 122 are designed to be magnetically coupleable with each other. That is, one or both of ICE system coupler 102 and/or needle system coupler 122 may include a magnetic portion. For example, in some embodiments ICE system coupler 102 is magnetic such that it exhibits a north pole 103 and a south pole 104. In some embodiments, complementary needle system coupler 122 includes a south pole 123 and a north pole 124. Accordingly, (i) north pole 103 of ICE system coupler 102 and (ii) south pole 123 of needle system coupler 122 attract each other. In addition, (a) south pole 104 ICE system coupler 102 and (b) north pole 124 of needle system coupler 122 attract each other. It should be understood that this is just one example arrangement, and any arrangement that results in a magnetic attraction between ICE system coupler 102 and needle system coupler 122 is within the scope of this disclosure (e.g., monopole arrangements). In some embodiments, one of either ICE system coupler 102 or needle system coupler 122 comprises a permanent magnet, while the other of either ICE system coupler 102 or needle system coupler 122 is merely comprises a magnetic material. The magnetic attraction forces between ICE system coupler 102 and needle system coupler 122 are strong enough to ensure secure attachment, while being not overly strong (thereby allowing safe disassembly to be performed).

In some embodiments, in addition to or as an alternative to magnetic attraction between ICE system coupler 102 and needle system coupler 122, ICE system coupler 102 and needle system coupler 122 may be configured to mechanically mate with each other. The mating may provide a releasable coupling therebetween, or merely a orientation therebetween. For example, in the depicted embodiment ICE system coupler 102 has an indentation and needle system coupler 122 has a corresponding protrusion. Accordingly, the indentation of ICE system coupler 102 can matingly receive the protrusion of needle system coupler 122 to provide a mechanical coupling therebetween. In some embodiments, ICE system coupler 102 and needle system coupler 122 may magnetically couple to each other without any mechanical coupling means (in terms of complementary indentations and protrusions, for example).

In some embodiments, one or more radiopaque markers may be disposed on either or both of ICE system 100 and trans-septal puncturing needle system 120. Such one or more radiopaque markers may be used to initiate and/or confirm the proper coupling orientation between ICE system 100 and trans-septal puncturing needle system 120 under fluoroscopy.

Referring also to FIGS. 4 and 5, ICE system 100 can be releasably coupled to trans-septal puncturing needle system 120. That is, ICE system coupler 102 of ICE system 100 can be releasably coupled with complementary needle system coupler 122 of trans-septal puncturing needle system 120. The coupling between ICE system coupler 102 and complementary needle system coupler 122 results in ICE system 100 being oriented in relation to trans-septal puncturing needle system 120 such that essentially all distal end portions of trans-septal puncturing needle system 120 are advantageously within the field of view of ICE system 100.

In some implementations, the coupling and subsequent decoupling between ICE system 100 and trans-septal puncturing needle system 120 can be performed while the distal end portions of ICE system 100 and trans-septal puncturing needle system 120 (including ICE system coupler 102 and complementary needle system coupler 122) are positioned within the body of the patient (e.g., within right atrium 14). Such coupling and subsequent decoupling may be performed under fluoroscopic visualization.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

What is claimed is:

1. A medical device system comprising:
an intra-cardiac echocardiography (ICE) system, wherein a distal end portion of the ICE system comprises an ICE system coupler; and
a second medical device, wherein a portion of the second medical device comprises a complementary coupler that is configured and operable to releasably couple with the ICE system coupler,
wherein coupling between the ICE system coupler and the complementary coupler results in an orientation therebetween that facilitates a desired in-situ visualization of the second medical device by the ICE system.

2. The medical device system of claim 1, wherein the ICE system coupler is attached to an ultrasonic probe of the ICE system.

3. The medical device system of claim 1, wherein the ICE system coupler is attached to a delivery sheath of the ICE system.

4. The medical device system of claim 1, wherein the second medical device comprises a trans-septal puncturing needle system.

5. The medical device system of claim 4, wherein the complementary coupler is attached to a needle of the trans-septal puncturing needle system.

6. The medical device system of claim 4, wherein the complementary coupler is attached to a delivery sheath of the trans-septal puncturing needle system.

7. The medical device system of claim 4, wherein, while the ICE system and trans-septal puncturing needle system are coupled together via the ICE system coupler and the complementary coupler, at least a distal tip of a needle of the trans-septal puncturing needle system is maintained in a position to be consistently visible by the ICE system.

8. The medical device system of claim 4, wherein, while the ICE system and trans-septal puncturing needle system are coupled together via the ICE system coupler and the complementary coupler, essentially an entire distal end portion of the trans-septal puncturing needle system is maintained in a position to be consistently visible by the ICE system.

9. The medical device system of claim 4, wherein the ICE system coupler and the complementary coupler are attracted to each other by magnetism.

10. The medical device system of claim 1, wherein the ICE system coupler and the complementary coupler are attracted to each other by magnetism.

11. The medical device system of claim 10, wherein one or both of the ICE system coupler and the complementary coupler comprise a permanent magnet.

12. The medical device system of claim 11, wherein the ICE system coupler and the complementary coupler comprise mating mechanical structures.

13. The medical device system of claim 1, wherein the ICE system coupler and the complementary coupler comprise mating mechanical structures whereby the ICE system coupler and the complementary coupler are releasably coupleable with each other.

14. A method for in-situ releasable coupling of an intra-cardiac echocardiography (ICE) system and a second medical device, the method comprising:
navigating, using an imaging modality, the ICE system such that a distal end portion of the ICE system is located within a target location within a patient, wherein a distal end portion of the ICE system comprises an ICE system coupler;
navigating, using an imaging modality, the second medical device such that a distal end portion of the second medical device system is located within the target location within the patient, wherein a portion of the second medical device comprises a complementary coupler that is configured and operable to releasably couple with the ICE system coupler; and
coupling, while the distal end portion of the ICE system and the distal end portion of the second medical device system are both located within the target location within the patient, the ICE system coupler with the complementary coupler such that the coupling results in an orientation therebetween that facilitates a desired in-situ visualization of the second medical device by the ICE system.

15. The method of claim 14, wherein the second medical device comprises a trans-septal puncturing needle system.

16. The method of claim 15, further comprising puncturing, using the trans-septal puncturing needle system, an interatrial septum of the patient while under direct visualization by the ICE system.

17. The method of claim 15, wherein the ICE system coupler and the complementary coupler are attracted to each other by magnetism.

18. The method of claim 14, wherein the ICE system coupler and the complementary coupler are attracted to each other by magnetism.

19. The method of claim 14, wherein the target location is a right atrium.

20. The method of claim 19, wherein the second medical device system comprises a trans-septal puncturing needle system, and wherein the method further comprises puncturing an interatrial septum with a needle of the trans-septal puncturing needle system while the trans-septal puncturing needle system and the distal end portion of the ICE system are magnetically coupled together.
